(19) United States
(12) Patent Application Publication
(10) Pub. No.: US 2016/0175144 A1
(43) Pub. Date: Jun. 23, 2016

(54) Multi-Spot Laser Probe With Molded Micro-Optical Glass Element

(71) Applicant: Novartis AG, Basel (CH)

(72) Inventors: Ronald T. Smith, Irvine, CA (US); Howell Schwartz, Lake Forest, CA (US)

(21) Appl. No.: 14/577,198

(22) Filed: Dec. 19, 2014

(51) Int. Cl. A61F 9/008

(52) U.S. Cl. CPC A61F 9/008

(57) ABSTRACT

An optical surgical probe can include a cylindrical cannula; a light guide, partially within the cannula, configured to receive a light beam from a light source through a proximal end, and to emit the light beam through a distal end; and a multi-spot generator at a distal end of the cannula, the multi-spot generator including a glass optical element with a faceted proximal surface, configured to receive the light beam from the light guide, and to split the light beam into beam-components; and a ball lens inside the glass optical element, configured to focus the beam-components to multiple spots in an image plane.

### BACKGROUND

1. Technical Field

This invention relates to an optical surgical probe and, more particularly, to a multi-spot laser surgical probe using a faceted optical element.

2. Related Art

Optical surgical probes deliver light to a surgical region for a variety of applications. In some applications, it may be useful to deliver light to multiple spots in the surgical region. For example, in pan-retinal photocoagulation of retinal tissue, an optical surgical probe that is configured to split a single laser or light beam into multiple beams focused to multiple retinal spots can cause photocoagulation at these spots simultaneously. Photocoagulating at n=2, 4, 6 spots simultaneously can reduce the time of the pan-retinal photocoagulation procedure approximately by a factor of 2, 4, or 6.

Various probe designs have been employed to produce multiple beams for a multi-spot pattern. For example, some probes include a diffractive element to divide a single beam into multiple beams corresponding to higher diffractive orders. Such diffractive elements are typically positioned inside the surgical probe, as positioning them at the end of the probe would pose substantial design challenges. However, positioning the diffractive elements away from the end of the probe can limit their functionalities.

Further, it has proven difficult to produce only the desired diffractive orders with high intensity while keeping the intensity of undesired diffractive orders sufficiently low. In addition, the performance of diffractive elements in different refractive media can differ substantially.

Another general challenge for the design of beam splitting probes is to fit them into a sufficiently small cannula at the end of the probe. The leading probes today have 23 Gauge cannulas, i.e. an outer diameter of about 0.650 mm. It is a non-obvious challenge to design the beam splitting elements to fit into these very narrow cannulas. Thus, a need persists for an optical surgical probe that can produce well-controlled multiple spots at a surgical target region using optical elements that can fit into the narrow cannula at the probe's end.

### SUMMARY

In particular embodiments of the present invention, an optical surgical probe can comprise a cylindrical cannula; a light guide, partially within the cannula, configured to receive a light beam from a light source through a proximal end, and to emit the light beam through a distal end; and a multi-spot generator at a distal end of the cannula, the multi-spot generator including a glass optical element with a faceted proximal surface, configured to receive the light beam from the light guide, and to split the light beam into beam-components; and a ball lens inside the glass optical element, configured to focus the beam-components to multiple spots in an image plane.

In some other embodiments, an optical surgical probe can comprise a cylindrical cannula; a light guide, partially within the cannula, configured to receive a light beam from a light source through a proximal end, and to emit the light beam through a distal end; and a multi-spot generator at a distal end of the cannula, the multi-spot generator including a glass optical element with a faceted proximal surface, configured to receive the light beam from the light guide, and to split the light beam into beam-components; wherein facets of the faceted proximal surface are curved to focus the beam-components to multiple spots in an image plane.

Other objects, features and advantages of the present invention will become apparent with reference to the drawings, and the following description of the drawings and claims.

### BRIEF DESCRIPTION OF THE DRAWINGS

FIG. 1 illustrates an optical surgical probe

FIG. 2 illustrates a perspective view of an optical surgical probe.

FIG. 3 illustrates an embodiment of the optical surgical probe without a ball lens.

### DETAILED DESCRIPTION

Some surgical probes split an incoming light beam, or laser beam, with a multi-spot generator that employs a faceted optical element to split the beam. The multi-spot generator can be positioned at the end of the cannula of the probe. It can also include a ball lens to focus the split beams. For ease of manufacture, the faceted optical element is typically made of an adhesive, which is cured after having been formed to the desired shape.

A problem with such probe designs is that for a variety of reasons the multi-spot generator can heat up when the beam is applied during the surgical procedure. In fact, a local operating temperature Tₒₚ of the multi-spot generator can rise close to or even beyond a critical temperature T_{c} of the cured adhesive: Tₒₚ>T_{c}. Here, the critical temperature T_{c} can reflect different undesirable physical processes that can have a negative or even critical effect on the performance of the probe. In some designs and applications, T_{c} can represent T_{c1}, the temperature when the cured adhesive gets detached from the cannula. In other cases, T_{c} can represent T_{c2}, the critical temperature where the cured adhesive becomes soft and malleable to a critical degree. T_{c2} is sometimes also called the soften temperature, or anneal temperature. Finally, in some cases, the critical temperature T_{c} can represent T_{c3}, the melting temperature of the cured adhesive. As is well known in the glass science and industry, the values of T_{c1}-T_{c3} depend to some degree on the details of their measurement and definition, so these temperatures are defined with a few percent tolerance. Any one of these physical processes can lead to the cured adhesive becoming soft or detached to a degree that the performance of the multi-spot generator deteriorates or even gets critically compromised.

In some cases, the performance deterioration can be gradual, such as the facets gradually change their shape thus causing the spots to form farther and farther from their intended location as the surgery proceeds. In other cases, the performance deterioration can be abrupt and critical, such as the ball lens getting loose and falling out at the end of the cannula into the eye chamber itself. Both the gradual and especially the critical performance deterioration are associated with the use of cured adhesives in these probes. Therefore, there is a need for optical surgical probes with multi-spot generators, whose faceted optical elements have critical temperatures T_{c} that are higher than the local operating temperature Tₒₚ of the probe. Such probes should not exhibit the just-described types of performance deterioration.

Recognizing this problem and positing a path to its solution has not been obvious, as it involved noticing that a systematic malfunction keeps recurring in the probes, properly identifying the cause of the malfunction among the numerous possibilities as the softening or melting of the cured adhesive, and discovering that materials and manufacturing processes with high enough T_{c} can be developed that solve the problem. Switching to a high T_{c} material is not a design choice either, because the manufacturing and manipulating high T_{c} materials is substantially more challenging than low T_{c} materials. Therefore, without having discovered the problem and that the path to its solution requires a high T_{c} material, a designer would have rather chosen a low T_{c} material instead.

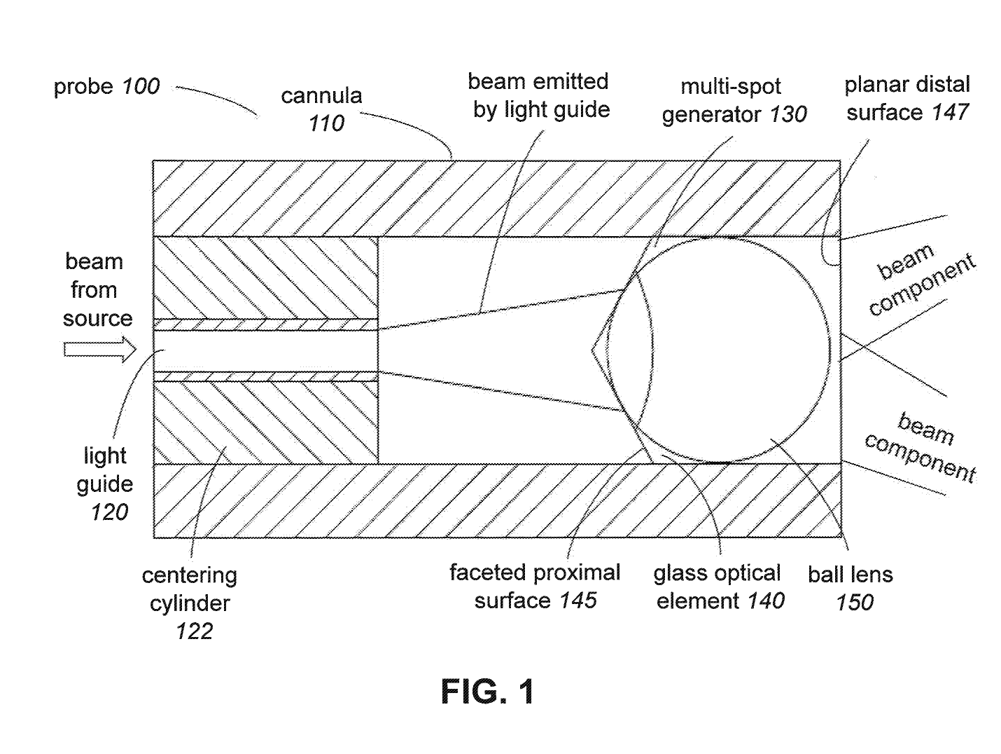

FIG. 1 illustrates embodiments that offer solutions for the above problems. An embodiment of an optical surgical probe 100 can include a cylindrical cannula 110 and a light guide 120 that is partially positioned within the cannula 110. The light guide 120 can be centered by a centering cylinder 122. The light guide 120 can be configured to receive a light beam from a light source through a proximal end, and to emit the light beam through a distal end. The light beam can be generated by a traditional incoherent light source, a light emitting diode (LED), or any coherent light source, such as a laser source. The wavelength of the light beam can take a wide variety of values. Direct tracking of the surgical procedure can be achieved for the surgeon by utilizing a light beam with a wavelength in the visible spectrum approximately in the 400-700 nm range. In some specific cases, the wavelength can be in the 500-600 nm range, such as 532 nm.

The light guide 120 can be an optical fiber, such as a glass fiber. The optical fiber can have a core-cladding-jacket structure. The light beam can be emitted by the light guide 120 with some degree of divergence. The divergent emitted light beam can have, for example, a numerical aperture NA in the range of 0.00-0.30, in some cases in the range of 0.10-0.20.

The light guide 120 can emit the light beam towards a multi-spot generator 130 that is positioned at a distal end of the cannula 110. The multi-spot generator 130 can be positioned at the very end of the cannula 110, its end flush with the cannula's end. In other embodiments, the multi-spot generator can be set back somewhat from the very end of the cannula 110.

Some embodiments of the multi-spot generator 130 can include an optical element 140 that is made of a high T_{c} critical temperature material, such as glass. The glass optical element can have a faceted proximal surface 145. The faceted proximal surface 145 can be configured to receive the light beam from the light guide 120, and to split, or to refract the light beam into beam-components. In various embodiments, the faceted proximal surface 145 can have 2, 4, 6 or other suitable number of facets. In some embodiments, the facets can be planar surfaces oblique to the optical axis of the cannula 110. The facets can make an angle with the optical axis in the 10-50 degrees range, in some cases in the 20-40 degrees range. In some embodiments, the facets can be non-planar, curved surfaces.

The multi-spot generator 130 can further include a ball lens 150 inside the glass optical element 140. The ball lens 150 can be fully inside the glass optical element 140. The ball lens 150 can be close to the faceted proximal surface 145, or to a planar distal surface 147 of the glass optical element 140, but can avoid touching or interrupting these surfaces to avoid disturbing the wavefronts. The ball lens 150 can be configured to focus the beam-components to multiple spots in an image plane. The focused beam components can be emitted through the distal surface 147 of the glass optical element 140, as shown. In an image plane, located at 1-8 mm from the distal end of the cannula 110, such as at 4 mm, the beam components can form spots, spaced apart by 0.5-2 mm. Embodiments of the faceted proximal surface 145 with 4 facets create 4 beam components that create 4 spots, for example arranged in a square. A suitably large surgical area can be covered or processed efficiently by placing these spot-squares in a repeating pattern.

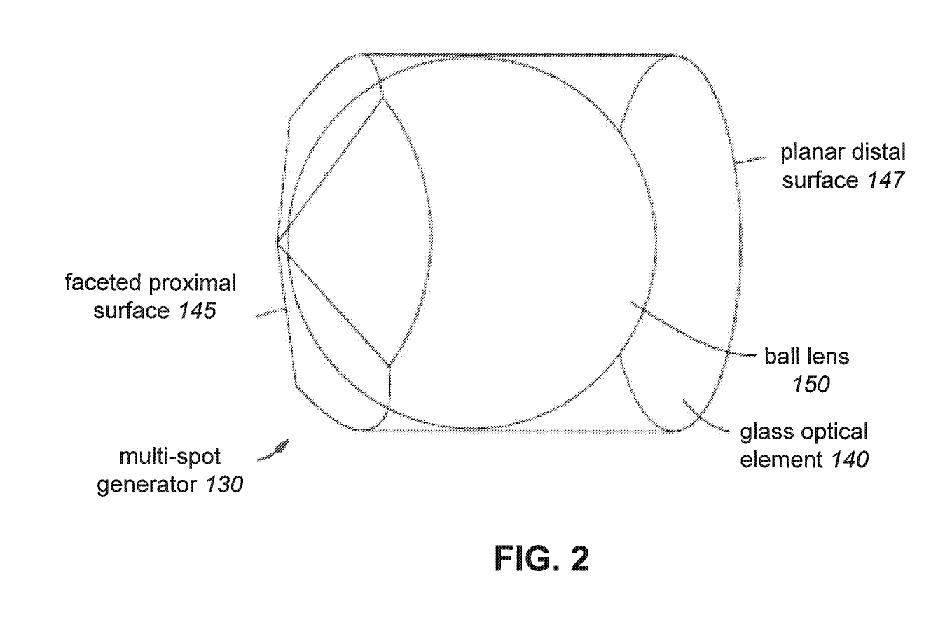

FIG. 2 illustrates an embodiment of the multi-spot generator 130 from a perspective view. As in FIG. 1, this embodiment of the multi-spot generator 130 can include the glass optical element 140 with the faceted proximal surface 145 and the planar distal surface 147. The glass optical element 140 can further include the ball lens 150.

The described embodiments of the optical surgical probe 100 address the above described problems by forming the optical element 140 from glass. As it is known, while various cured adhesives typically have critical temperatures T_{c} in the few hundred centigrade range, such as T_{c}(adhesive)˜200-300 C (centigrade), large classes of glass have critical temperatures in the T_{c}(glass)˜500-1,000 C range. At the same time, the local operating temperature Tₒₚ of surgical probes is often in the 100-300 C range. Therefore, optical surgical probes 100 that include an optical element 140 that is made of glass instead of a cured adhesive are capable of operating at a local operating temperature Tₒₚ that is lower than a critical temperature T_{c} of the optical element with a sufficient margin to avoid the described undesirable performance deterioration. Here, Tₒₚ refers to a local operating temperature of the glass optical element 140 itself, as there can be substantial temperature gradients throughout the surgical optical probe 100, and the critical temperature T_{c} is one of a temperature T_{c1} where the glass optical element 140 detaches from the cannula 110, a temperature T_{c2} where the glass optical element 140 becomes soft to a critical degree, and a melting temperature T_{c3} of the glass optical element 140.

In some embodiments, a critical temperature T_{c} of the glass optical element 140 is higher than 500 centigrade: T_{c}>500 C. Examples of such glass optical elements 140 include molded glass.

Concerning the optical characteristics of the glass of the optical element 140, an index of refraction of the glass optical element 140 can be between 1.4 and 1.6 in some embodiments.

In some embodiments of the optical surgical probe 100 the cannula 110 can be sized to be 23 Gauge or smaller. This translates to the cannula 110 having an outer diameter less than 700 microns and an inner diameter less than 400 microns. In some embodiments, the cannula has an outer diameter of 650 microns plus minus 10 microns.

In some embodiments, a diameter of the ball lens 150 can be between 100 and 500 microns. In some embodiments, the diameter of the ball lens 150 can be between 350 and 400 microns.

In some embodiments, the ball lens 150 can comprise sapphire. An index of refraction of the ball lens can be in the 1.55-1.85 range, in some embodiments in the 1.7-1.8 range.

In some embodiments of the probe 100, the glass optical element 150 comprises molded glass.

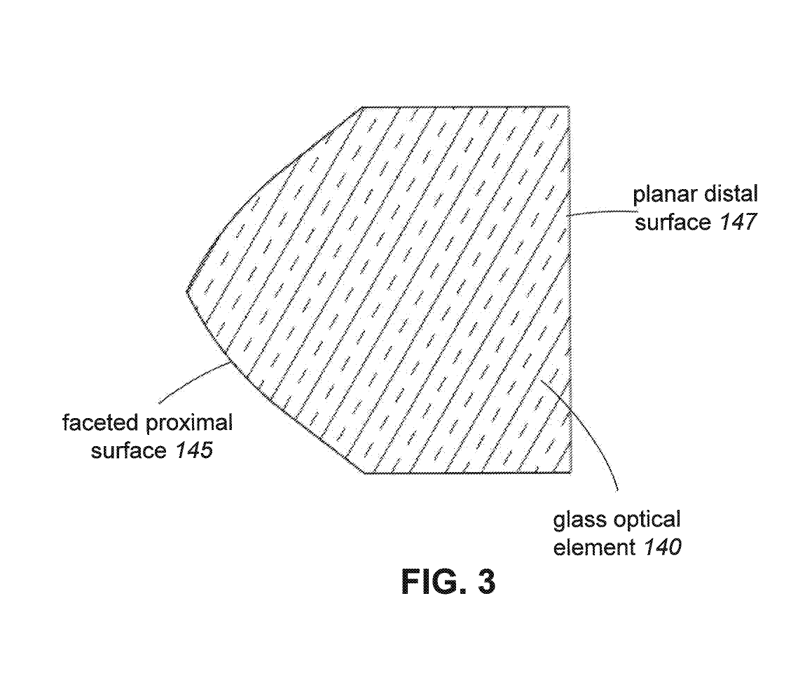

FIG. 3 illustrates that in some embodiments, the glass optical element may not include a ball lens 150. In such embodiments, the glass optical element 140 can still include a faceted proximal surface 145. In these embodiments, the facets may be curved. Curved facets can not only split the incident beam into beam components, but also focus the beam components to spots in an image plane.

The present invention is illustrated herein by example, and various modifications may be made by a person of ordinary skill in the art. Although the present invention is described in detail, it should be understood that various changes, substitutions and alterations can be made hereto without departing from the scope of the invention as claimed.

1. An optical surgical probe comprising:
a cylindrical cannula;
a light guide, partially within the cannula, configured
to receive a light beam from a light source through a proximal end, and
to emit the light beam through a distal end; and
a multi-spot generator at a distal end of the cannula, the multi-spot generator including
a glass optical element with a faceted proximal surface, configured
to receive the light beam from the light guide, and
to split the light beam into beam-components; and
a ball lens inside the glass optical element, configured to focus the beam-components to multiple spots in an image plane.

2. The optical surgical probe of claim 1, wherein:
a local operating temperature of the multi-spot generator is lower than a critical temperature of the glass optical element.

3. The optical surgical probe of claim 2, wherein:
the critical temperature is one of a temperature where the glass optical element detaches from the cannula, a temperature where the glass optical element becomes soft to a critical degree, and a melting temperature of the glass optical element.

4. The optical surgical probe of claim 2, wherein:
the critical temperature of the glass optical element is above 500 centigrade.

5. The optical surgical probe of claim 1, wherein:
the glass optical element comprises molded glass.

6. The optical surgical probe of claim 1, wherein:
an index of refraction of the glass optical element is between 1.4 and 1.6.

7. The optical surgical probe of claim 1, wherein:
the cannula is sized to be 23 Gauge or smaller.

8. The optical surgical probe of claim 7, wherein:
an outer diameter of the cannula is less than 700 microns and an inner diameter of the cannula is less than 400 microns.

9. The optical surgical probe of claim 1, wherein:
a diameter of the ball lens is between 100 and 500 microns.

10. The optical surgical probe of claim 9, wherein:
the diameter of the ball lens is between 350 and 400 microns.

11. The optical surgical probe of claim 1, wherein:
the ball lens comprises sapphire.

12. The optical surgical probe of claim 1, wherein:
the faceted proximal surface has four facets.

13. The optical surgical probe of claim 1, wherein:
the light guide is held in place by a centering cylinder.

14. An optical surgical probe, comprising:
a cylindrical cannula;
a light guide, partially within the cannula, configured
to receive a light beam from a light source through a proximal end, and
to emit the light beam through a distal end; and
a multi-spot generator at a distal end of the cannula, the multi-spot generator including
a glass optical element with a faceted proximal surface, configured
to receive the light beam from the light guide, and
to split the light beam into beam-components; wherein
facets of the faceted proximal surface are curved to focus the beam-components to multiple spots in an image plane.
